(19) United States
(12) Patent Application Publication
(10) Pub. No.: US 2007/0279234 A1
(43) Pub. Date: Dec. 6, 2007

(54) Pediatric sleep trainer

(76) Inventor: Nancy Walsh, Los Altos, CA (US)

Correspondence Address: Joseph P. Quinn, Esq.;Brown Rudnick Berlack Israels LLP, One Financial Center, Boston, MA, 02111, US

(21) Appl. No.: 11/443,725

(22) Filed: May 31, 2006

(51) Int. Cl. G08B 23/00; G08B 1/00; G08B 1/08; A61M 21/00

(52) U.S. Cl. 340/573.1; 340/309.16; 340/539.1; 600/27

(57) ABSTRACT

A system and method for training young children to return to sleep when they wake without requiring a parent to come to their side. Illustrative embodiments of the invention help eliminate night awakenings, ensure consistent naptime, establish a bedtime routine and allow a caregiver to set an infant or toddler's sleep schedule. Illustrative embodiments use a visual stimulus, such as a light, to create a strong association with appropriate sleep behaviors and to provide information to the child as to whether it is time to get up or continue sleeping.

### BACKGROUND OF THE INVENTION

Young children, especially infants and toddlers, frequently wake from their sleep during the night and call out for a parent. Parents are generally compelled to go to the child's side to console the child so that the child will go back to sleep. Sometimes a child is difficult to console or will have difficulty going back to sleep. Other times a child may go quickly to sleep once a parent checks in and tells the child to do so. However, even when a child goes quickly back to sleep, the parent may have difficulty going back to sleep after being awakened by the child.

Similarly, infants and toddlers often wake in the morning at an unacceptably early hour. When parents try to shift a child's sleep pattern by putting the child to bed later having hope that the child will sleep later, the child will generally wake at the same early hour. Furthermore, the child's reduced sleep time will typically cause the child to be tired and irritable in the morning.

A primary cause for young children's inability to put themselves back to sleep is their lack of time perception. Because young children cannot tell time, have no sense of when they are tired, and have no sense of the passage of time, they do not know when they should still be sleeping. Accordingly, rather than returning to sleep, a young child who awakens during the night will typically call or cry for a parent.

### SUMMARY OF THE INVENTION

The purpose of the present invention is to train infants and toddlers to return to sleep when they wake at night without requiring a parent to come to their side. Illustrative embodiments of the invention help eliminate night awakenings, ensure consistent naptime, establish a bedtime routine and allow a caregiver to set an infant or toddler's sleep schedule. Illustrative embodiments use a visual stimulus, such as a light, to create a strong association with appropriate sleep behaviors and to provide information to the child as to whether it is time to get up or continue sleeping.

Infants and toddlers benefit from a bedtime routine that provides consistent cues which they can associate with sleeping. Many sleep experts recommend providing a child with various sleep cues such as by consistently providing some soothing sounds or dimming lights at the child's sleep time to help the child go to sleep. However, these techniques do not give the child cues to return to sleep when they wake up during the night. These techniques also do not help the child to know when it is time to get up.

Illustrative embodiments of the present invention can train a child to associate a correct time to get up with a particular cue. In a particular embodiment, a child can be trained to associate a light coming on or going off with the proper time to get up. Various embodiments of the invention provide an easy to use, portable cue for going to sleep, and for knowing whether or not it is time to get up when a child awakens.

Illustrative embodiments of the present invention provide a sleep training system including a cueing portion disposed within an effective range of a child's sleep place. A timer is provided in communication with the cueing portion and configured to enable and disable the cueing portion at preselected times. A remote controller can be provided in communication with the timer and configured to remotely override the timer.

In an illustrative embodiment, the cueing portion provides a visible cue such as a light that a child can associate with sleep at times indicated by the timer. In a particular embodiment, the light can be configured to gradually dim at a sleep time indicated by the timer and wherein the light is configured to gradually brighten at a wake time indicated by the timer. The lamp can be a child's table lamp with a timer incorporated therein or the lamp can be incorporated in a child's toy or doll, for example.

In another embodiment, the cuing portion provides an audible cue that a child can associate with sleep at times indicated by the timer. For example, the cuing portion can be an annunciator configured to emit sounds at times indicted by the timer. The annunciator could be incorporated within a child's toy, doll or stuffed animal for example.

In another embodiment, the cuing portion can include an animated object having a movable portion configured to move from a first position to a second position at times indicated by the timer. For example, the animated object can be a doll, stuffed animal or the like wherein the movable portion can emulate eyelids configured to open and close at the times indicated by the timer. In another example, the animated object could be a doll configured to lay down at sleep times indicated by the timer and to arise at wake times indicated by the timer. In still another embodiment, the cuing portion can include automatically actuated window blinds configured to close at a sleep time indicated by the timer and open at a wake time indicated by the timer.

In an illustrative embodiment of the invention, the timer can incorporated into an electrical outlet such that the cuing device can be plugged into the outlet to receive alternating current power therefrom at times indicated by the timer. In an alternative embodiment, the timer can be incorporated into a lamp socket wherein the cueing device comprises a lamp installed in the socket.

### BRIEF DESCRIPTION OF THE DRAWINGS

The foregoing and other features and advantages of the present invention will be more fully understood from the following detailed description of illustrative embodiments, taken in conjunction with the accompanying drawings in which:

FIG. 1 is a pictorial diagram of a sleep training system according to an illustrative embodiment of the present invention;

FIG. 2 is a process flow diagram of a method for using the sleep training system with an untrained child according to an illustrative embodiment of the present invention; and

FIG. 3 is a process flow diagram of a method for using the sleep training system with a trained child according to an illustrative embodiment of the invention.

### DETAILED DESCRIPTION

Illustrative embodiments of the present invention provide a signal that lets young children know when it was time to get up, and when it is time to go back to sleep so that they do not call a parent or caregiver during their sleep time or during naptimes.

Illustrative embodiments of the present invention take advantage of certain attributes of young children's sleep habits to modify children's sleep patterns. For example, it has been recognized that sleep habits are learned behaviors and that many children who have sleep problems, have not learned the appropriate sleep behaviors. Most people wakes up at times during the night, however because young children can not recognize when they are tired, they do not know when they should sleep more. It has also been heretofore known that young children do not have any sense of time to help them establish sleep habits.

It has been recognized that a consistent sleep routine helps children go to sleep. To establish a sleep routine, young children recognize various cues in their external environment. These cues can be manipulated to modify a child's sleep patterns but must be provided consistently to the child. In an illustrative embodiment of the invention, inanimate objects can be used to provide cues to aid in establishing a child's sleep routine because children believe that inanimate objects hold great power. A remote controller can be used to ensure that the cue is consistently provided at the proper time relative to a child's behavior. Consistency particularly during the training phase because the cue can be tailored to the behavior of the child. This allows a child, especially a preverbal child, to make the association sooner.

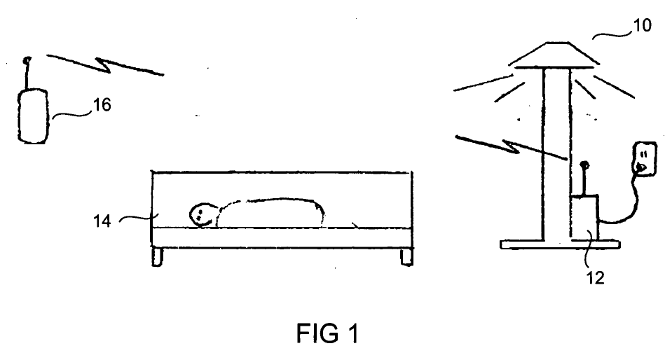

An illustrative embodiment of the present invention is described with reference to FIG. 1 in which a cuing device 10 is enabled by a timer 12 incorporated therewith. The cuing device 10 is placed in effective range of the child's sleep place 14. The timer 12 can be set to enable a sleep cue at a child's predetermined sleep time, and to disable the sleep cue (or enable a wake cue) at the child's predetermined wake time. The timer can be over-ridden by a signal from a remote controller 16 which allows a parent change the sleep cue's state at a arbitrary times, for example when it is desired to allow a child to get up at an unscheduled time so that the child can still receive the wake up cue from the cueing device. In the illustrative embodiments, in order to strengthen the child's association with the sleep cue, the child should not be informed that the parent or care giver has any control over the cuing device.

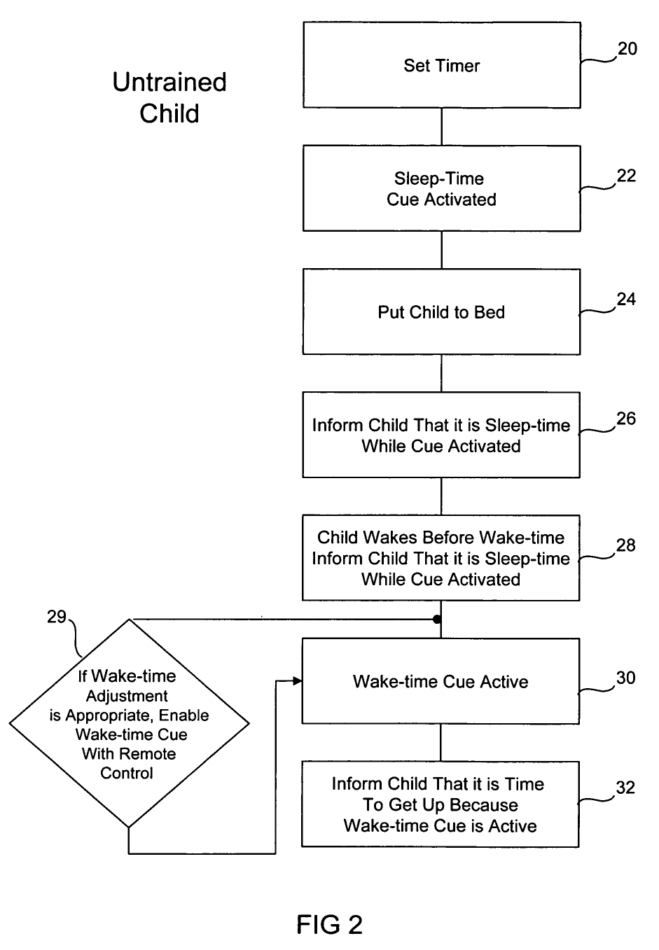

A method of using the sleep training system with an untrained child according to an illustrative embodiment of the invention is described with reference to FIG. 2. During a preliminary step 20 a parent or caregiver sets a timer to enable a sleep time cue at a the child's predetermined sleep time and disable the sleep time cue (or enable a wake cue) at the child's predetermined wake time. When the child's sleep time occurs, a sleep time cue is activated 22 and provides a cue within range of the child's sleep place so the child can learn to associate the cue with sleep. The child can be put to bed 24 just when the sleep time cue is enabled. Alternatively, a child can be put to bed just before the sleep time cue is enabled so that the child can witness the sleep time cue change state. In either case, the child can should be informed 26 that it is sleep time when the sleep time cue is enabled. If a child wakes before the predetermined wake time, the child should be informed 28 that she can not get up because the sleep time cue is still enabled. If a waketime adjustment is appropriate, the waketime cue can be enabled with a remote control 29. When wake time occurs, the sleep time cue is disabled or wake time cue is enabled 30. The child should be informed 32 that she can get up because the wake time cue is enabled 30.

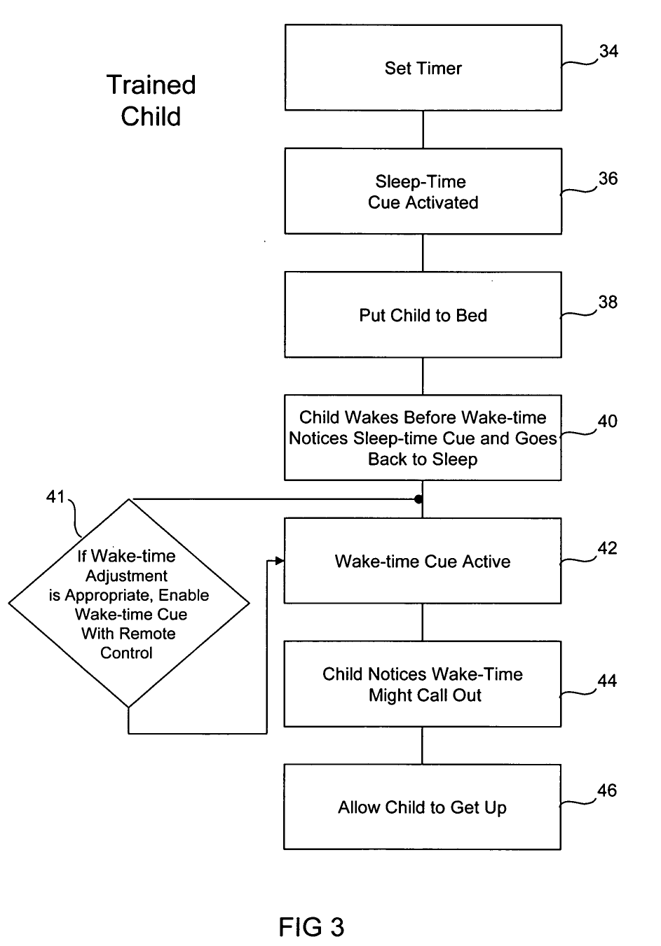

A method of using the sleep training system with a trained child according to an illustrative embodiment of the invention is described with reference to FIG. 3 During a preliminary step 34 a parent or caregiver sets a sleep timer to enable a sleep time cue at the child's predetermined sleep time and disable the sleep time cue (or enable a wake time cue) at the child's predetermined wake time. When the child's sleep time occurs, a sleep time cue is activated 36 and provides a cue with range of the child's sleep place so the child can reinforce the association of sleep with the cue when the child is put to bed 38. If the child wakes before the predetermined wake time 40, the child will notice the sleep time cue and return to sleep. When the predetermined wake time occurs 42, the child may notice the wake time cue and call out for a parent 44. If a waketime adjustment is appropriate, the waketime cue can be enabled with a remote control 41. The parent can allow the child to get up 46 only when the sleep time cue is disabled or the wake time cue is enabled.

In an illustrative embodiment, the signal which a child learns to associate with sleep is a light that can be seen by the child from bed. Once the child associates an on or off condition of the light with sleep, a parent can use the light to change the time the child wakes up in the morning. For example, a parent can adjust a child's sleep time by putting the child to bed a little later each night and adjusting the light to go off a little later each day. Thus, when the child wakes up at her normal time, which is earlier than the parent would like, the child can see the light and recognize that it is not yet time to get up. Because the child has been put to bed later, she will typically still be tired and will go back to sleep rather than of calling for a parent.

In an illustrative embodiment, a nightlight can be attached to a digital timer and placed in view of a child's bed. The child can be trained not to get up until the light came on, for example, by setting the timer to turn on the light a few minutes later than the child usually gets up. When the child wakes up, the parent can train the child by pointing to the light and telling the child that it is not time to get up until the light goes on. As soon as the timer turns the light on, the parent can train the child by pointing to the light, telling the child that the light was on then immediately taking the child out of her bed.

A particular embodiment of the invention includes a digital timer which is remotely controllable. Because consistency is very important when training a child to change her behavior, it is important to make the child wait for the light to go on before allowing the child to get up or before the parent appears to the child. However, it is occasionally desirable for a parent or care giver to allow a child to get up earlier than a previously intended time. The child should not be allowed to see a parent controlling the light because the child would then simply cry for the parent to turn on the light. Accordingly, an illustrative embodiment of the invention provides a remotely controllable timer for a child's light. Rather than standing outside of a child's view while waiting for the light to come on, a parent wishing to allow the child to arise early can use the remote control to bypass the timer function and turn on the light without letting the child know that the parent has control of the light.

According to illustrative embodiments of the invention, within about one week a child can be trained to understand that they should only get up when the light was on. Such understanding generally will stop the child from waking a parent during the night due to normal night awakenings. After about a week of such training, sleep times can be adjusted, for example by putting the child to bed 15 minutes later and setting the timer to turn the light on 15 minutes later. Using this method according to the illustrative embodiment of the invention, over a period of about 3 weeks a child's wake time can be adjusted from 5:00 A.M. to getting up about 7:00 A.M., for example.

By using illustrative embodiments of the present invention, children can form a strong belief that it is improper to get up until the light comes on. If a child does wake up before the light comes on, they generally will not ask a care giver to turn on the light on because they believe that the care giver has nothing to do with the light. The illustrative embodiment of the present invention provides additional safety because children who are trained to stay in bed until the light comes on will also be less likely to climbed out of their cribs or beds during the night. Illustrative embodiments of the invention can also be used to help children go to sleep and as to stay asleep at bedtime and naptime.

An illustrative embodiment of the present invention includes a light or other visual stimulus controlled by both a programmable timer and a wireless remote control. The timer can be set to make the light or other visual stimulus go on when the young child should get up and go off when the child should go to sleep. In an illustrative embodiment, the timer can include a count down function and/or countdown notification portion. Embodiments of the invention provide a consistent sleep cue to teach young children desired sleep behaviors and can inform children when it is time to get up.

In an illustrative embodiment, light from a nightlight, table lamp or other visual stimulus can provide a child with avery good sleep association if it can be seen by the child regardless of the time of night. For example, a child who wakes up during the night, or very early in the morning can see that the light is not on and can understand that it is therefore not time to get up. The child generally can go back to sleep on their own rather than calling for a caregiver to come in and tell them to go back to sleep. Additionally a child cannot argue or plead with an inanimate object, making it an effective and consistent sleep cue.

Generally, the timer will be set to turn a light on at the time the child normally get up. However, the child may awake before the timer goes off. During training, the caregiver can go in and tell the child it is not wake up time. This should be repeated until the timer causes the light to go on.

On occasion, a child will wake up many minutes before the light is scheduled to go on. The child's caregiver may not want to have to repeatedly tell the child to stay in the crib. By using the remote control the caregiver can override the timer and turn on the light maintaining the consistency of having the child stay in the crib until the light goes on without having to repeat the message until the light comes on via the timer. The remote control is important because the child must not associate the light's state with the caregiver's activities. Thus the remote control can facilitate consistency in the training and can remove the cue from the apparent control of the caregiver.

In a particular illustrative embodiment of the invention, the timer can be controllable to allow the light to dim as a child's sleep time approaches. In another particular embodiment, the light source may be combined in a stuffed animal or other object that can be taken to bed with the child.

In another illustrative embodiment of the invention a stimulus other than light may be used as a cue for the child to associate with sleep time. For example, the stimulus may be the face of a toy person or animal who's eyes close to indicate a child's sleep time and open to indicate the child's wake time. In another illustrative embodiment, the invention can include an toy animal or doll which is automated to get into bed or lie down when it is the child's sleep time and get up from bed or sit up when it is the child's wake time. Because children respond well to human faces, an illustrative embodiment of the invention can provide a lamp incorporated with a model human face to light the face according to a timer and thereby provide a strong cue for a child to associate with sleep.

Although the invention has been shown and described with respect to exemplary embodiments thereof, various other changes, omissions and additions in the form and detail thereof may be made therein without departing from the spirit and scope of the invention.

What is claimed is:

1. A sleep training system comprising:
a cueing portion disposed within an effective range of a child's sleep place;
a timer in communication with said cueing portion, said timer being configured to enable and disable said cueing portion at preselected times; and
a remote controller in communication with said timer.

2. The system according to claim 1 wherein said remote control is configured to remotely override said timer.

3. The system according to claim 1 wherein said cueing portion provides a visible cue that a child can associate with sleep at times indicated by said timer.

4. The system according to claim 1 wherein said cueing portion provides light at times indicated by said timer.

5. The system according to claim 4 wherein said light is configured to gradually dim at a sleep time indicated by said timer and wherein said light is configured to gradually brighten at a wake time indicated by said timer.

6. The system according to claim 1 wherein said cueing portion provides an audible cue that a child can associate with sleep at times indicated by said timer.

7. The system according to claim 1 wherein said cuing portion comprises a toy having a lamp portion incorporated therewith wherein said lamp portion provides light at times indicated by said timer.

8. The system according to claim 1 wherein said cuing portion comprises a lamp with said remote timer incorporated therewith wherein said lamp provides light at times indicated by said timer.

9. The system according to claim 1 wherein said cuing portion comprises an annunciator configured to emit sounds at times indicted by said timer.

10. The system according to claim 1 wherein said cuing portion comprises a toy having an annunciator portion incorporated therewith wherein said annunciator portion provides sounds at times indicated by said timer.

11. The system according to claim 1 wherein said cuing portion comprises an animated object having a movable portion configured to move from a first position to a second position at times indicated by said timer.

12. The system according to claim 11 wherein said animated object comprises a doll and wherein said movable portion comprises are eyelids configured to open and close at said times indicated by said timer.

13. The system according to claim 10 wherein said animated object comprises a doll configured to lay down at sleep times indicated by said timer and to arise at wake times indicated by said timer.

14. The system according to claim 1 where said cuing portion comprises automatically actuated window blinds configured to close at a sleep time indicated by said timer and open at a wake time indicated by said timer.

15. The system according to claim 1 wherein said timer is incorporated into an electrical outlet wherein said cuing device can be plugged into said outlet to receive alternating current power therefrom at times indicated by said timer.

16. The system according to claim 1 wherein said timer is incorporated into a lamp socket wherein said cueing device comprises a lamp installed in said socket.

17. A method for training a child to sleep comprising:
providing a cuing device in effective range of the child's sleep place; and
setting a remotely overridable timer to enable said cuing device at said child's sleep time and disable said cuing device at said child's scheduled wake time, wherein said cuing device when enabled provides a signal for association with sleep to the child, and when disabled provides a signal for association with waking up to the child.

18. The method according to claim 17 further comprising informing said child that they can not get up when awakened if the cueing device is enabled.

19. The method according to claim 17 further comprising remotely overriding said timer to disable said cuing device when said child is allowed to get up before a preset wake time, or to enable said cuing device when said child is allowed to go to bed before a preset sleep time.

20. The method according to claim 17 further comprising progressively reducing a caregiver's response to child who cries during scheduled sleep times as said child's association of sleep with said cuing device increases.
